(19) United States
(12) Patent Application Publication
(10) Pub. No.: US 2008/0268022 A1
(43) Pub. Date: Oct. 30, 2008

(54) MEHTODS FOR TREATING AND PREVENTING AILMENTS CAUSED BY HUMAN PAPILLOMAVIRUS

(76) Inventors: R. Tyler McCabe, North Salt Lake, UT (US); Donald F. Grabarz, Salt Lake City, UT (US); Duck Joo Lee, Holladay, UT (US)

Correspondence Address: KENNETH E. HORTON;KIRTON & MCCONKLE, 60 EAST SOUTH TEMPLE, SUITE 1800, SALTLAKE CITY, UT, 84111, US

(21) Appl. No.: 12/036,164

(22) Filed: Feb. 22, 2008

Related U.S. Application Data
(60) Provisional application No. 60/891,330, filed on Feb. 23, 2007.

(51) Int. Cl. A61K 31/731; A61K 9/70; A61K 9/00; A61P 31/12

(52) U.S. Cl. 424/430; 514/54; 424/449

(57) ABSTRACT

This application discusses methods and compositions for preventing and treating ailments and/or diseases, such as HPV, with carrageenan. Moreover, methods and systems for administering carrageenan are described herein. In some embodiments, carrageenan (i.e., carrageenan, carrageenan prodrugs, carrageenan metabolites, carrageenan derivatives, and so forth) may be used prophylacticly in order prevent and/or slow a subject from receiving an ailment and/or disease. In other embodiments, carrageenan may be used to treat, suppress, and/or ameliorate an ailment and/or disease after a subject has a condition. While carrageenan may be administered alone, in some cases, the beneficial effects of carrageenan can be synergistically or additively improved by co-administering carrageenan with a polyamide, a polyamide-like compound, and/or surfactant. While the active ingredient(s) may be administered in any form, some preferred forms may include the use of a gel, foam, spray, solution or an intravaginal ring comprising the active ingredient(s).

### FIELD

This application relates generally to methods for treating and preventing disease and/or ailments. In particular, this application relates to methods for treating and preventing ailments and/or diseases with an effective amount of any suitable type of carrageenan, carrageenan derivative, carrageenan prodrug, carrageenan metabolite, polyamide, polyamide-like compound, surfactant agent, or combination thereof and administration, application, and delivery

### BACKGROUND

Papillomaviruses infect some skin and mucosal tissues of many vertebrate species, including humans. There are approximately 100 known genotypes of human papillomavirus (HPV). A subset of papillomaviruses, genital mucosotropic human papillomaviruses, cause almost all cases of cervical cancer, which is the second most common cancer in terms of both incidence and mortality worldwide. Such HPVs also cause a substantial fraction of head-and-neck and ano-genital cancers. Additionally, some HPVs are associated with benign genital warts.

Some estimate that the average person has a seventy-five percent risk of HPV infection during a lifetime. Despite the fact that most HPV infections are subclinical and self-limiting, some of those infected develop lesions, which can become premalignant or cancerous. Although HPV vaccines are available to the public, such vaccines can be expensive, may be limited to the treatment of certain types of papillomaviruses, and can have undesirable side effects, systemic or otherwise.

Recently, scientists have discovered that carrageenan, a sulfated polysaccharide found in red algae (e.g., seaweed), can be a potent inhibitor of infections caused by HPVs, as well as herpes simplex viruses and Human Immunodeficiency Virus (HIV). In a non-binding theory, it is believed that carrageenan acts to prevent HPV virons from binding to cells. This theory is bolstered by the fact that carrageenan closely resembles heparin sulfate, which is a HPV cell-attachment factor. In this theory, carrageenan may chemically mimic heparin sulfate and thereby compete against the viron for initial attachment to the surface of a cell. Additionally, it is theorized that carrageenan can inhibit HPV infection through another way involving a post-attachment heparin sulfate-independent effect. A more complete explanation of carrageenan and its mechanisms of viron infection inhibition can be found in Christopher B. Buck et al., Carrageenan is a Potent Inhibitor of Papillomavirus Infection, PLOS PATHOGENS, vol. 2, iss. 7, 0671, 0671-80 (July 2006).

Carrageenan is currently used in many cosmetic products and foods as a thickener. Nevertheless, methods of carrageenan administration and its uses may currently be somewhat limited.

### SUMMARY

This application discusses methods, compositions, formulations, and systems for preventing and treating ailments and/or diseases, such as HPV, with carrageenan. Moreover, methods and systems for administering carrageenan are described herein. In some embodiments, carrageenan (i.e., carrageenan, carrageenan prodrugs, carrageenan metabolites, carrageenan derivatives, and so forth) may be used prophylacticly in order prevent and/or slow a subject from receiving an ailment and/or disease. In other embodiments, carrageenan may be used to treat, suppress, and/or ameliorate an ailment and/or disease after a subject has a condition.

In some cases, carrageenan may be co-administered with other agents that can further prevent and/or treat an ailment or disease. In some instances, these agents may act synergistically or additively with carrageenan to increase the effectiveness of carrageenan. Some non-limiting examples of such agents may include polyamides, polyamide-like compounds, surfactant agents, and/or combinations thereof.

Carrageenan and any suitable agent may be administered to a subject in virtually any suitable way that is conducive to the treatment, suppression, prevention, and/or amelioration of an ailment or disease, including, but not limited to vaginally, rectally, transurethrally, mucosally, topically, transdermally, etc.

Similarly, carrageen and a suitable agent can be administered to a subject in any way that is conducive to the treatment, suppression, prevention, and/or amelioration of an ailment or disease. For example, carrageenan can be administered in the form of a gel (e.g., aqueous or solid/soluble), a cream, a foam, a spray, an intrauterine device, or an intravaginal ring (IVR). In some embodiments, it may be beneficial to administer carrageenan in a form that releases carrageenan and any co-administered agent in a controlled, continuous, and/or time release manner. For example, when carrageenan is administered through the use of an IVR, the carrageenan formulation can remain active for a period up to about 30 days or longer.

In some cases, carrageenan and a suitable agent, if included, can be co-administered in conjunction with any known pharmaceutical and/or nutraceutical and may be used with and administered with any known system, apparatus, and/or material.

### BRIEF DESCRIPTION OF THE DRAWINGS

The following description can be better understood in light of Figures, in which:

FIG. 1 contains a drawing illustrating some embodiments of carrageenan;

FIG. 2 contains a drawing illustrating some embodiments of carrageenan; and

FIG. 3 contains a drawing illustrating some embodiments of carrageenan.

Together with the following description, the Figures help to demonstrate and explain the methods and systems for using and administering carrageenan.

### DETAILED DESCRIPTION

The following description supplies specific details in order to provide a thorough understanding. Nevertheless, the skilled artisan would understand that the described methods and associated compositions, materials, apparatus, chemicals, etc. can be implemented and used without employing these specific details. Indeed, the methods and associated compositions, materials, apparatus, etc. can be placed into practice by modifying the methods and associated compositions and can be used in conjunction with any composition, material, apparatus, chemical, compound, solution, technique, etc. conventionally used in the pharmaceutical or medical device industry.

Generally, this application discusses methods and systems for administration and treatment of a subject for one or more ailments and/or diseases with carrageenan. As used herein, the term treatment includes preventative (e.g., prophylactic) and palliative treatment. Also, as used herein, the term treating can include the act of providing preventative, curative, and/or palliative treatment. Moreover, the described methods of treatment can comprise the administration of any amount, including an effective amount, of carrageenan to a subject. In such a manner, the described methods can be used to prevent, treat, suppress, and/or ameliorate an ailment and/or disease (hereinafter “condition).

The described methods may be used for the treatment of any type of subject. For example, a suitable subject may comprise any form of life. In some instances, a suitable subject may include one or more cells, tissues, organs, systems, and/or organisms. For example, a suitable subject may comprise an animal, such as a human. Further, a suitable subject may be a male or female, depending on the condition being treated and/or prevented.

While the description below focuses on using carrageenan and/or materials, apparatus, compositions, devices, and the like comprising carrageenan, for the use of treating a condition caused by any form of human papillomavirus, such as a cervical cancer, the methods and systems described herein may be useful for the treatment of any condition. As an example, the described methods and systems may be effective against conditions such as various forms of genital warts, herpes, HIV, cytomegalovirus, syphilis, gonorrhea, hepatitis, mulluscum, dengue, warts, cold sores, cankers, cancers (e.g., carcinomas, malignancies, ano-genital cancers, colon cancer, uterine cancer, ovary cancer, cervical cancer, sarcomas, melanomas, renal cell carcinoma, etc.), autoimmune diseases, alopecia, endometriosis, uterine fibrosis, atrophic vaginitis, dysparcunia, vagismus, changing vaginal pH, and so forth.

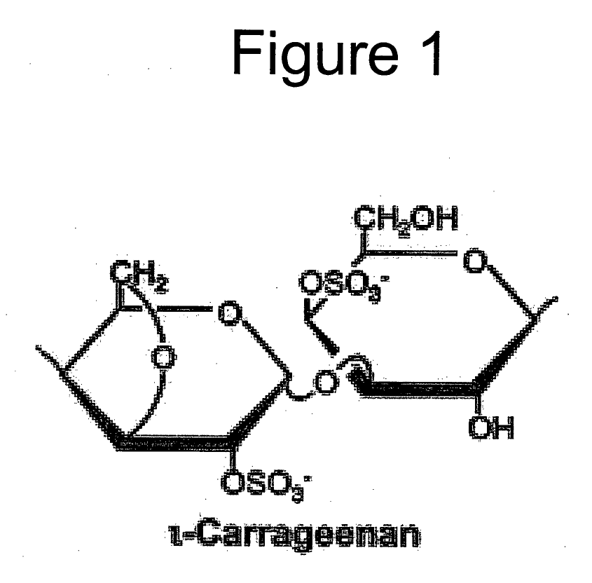

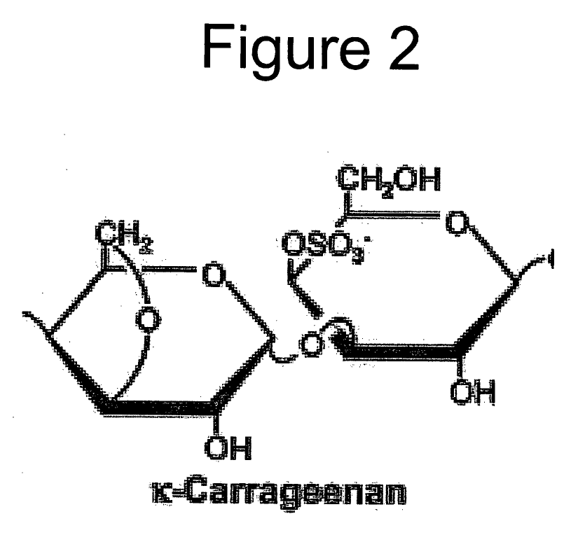

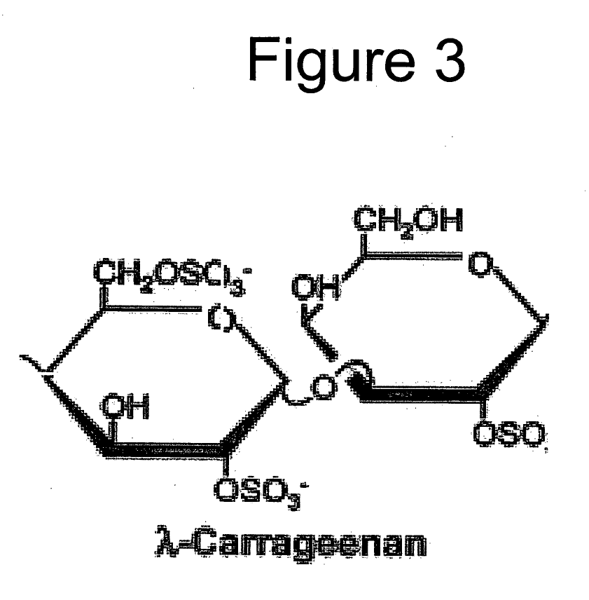

In some embodiments, any type or combination of different types of carrageenan and/or carrageenan derivative can be used for the described methods of treatment for a condition. For example, FIGS. 1, 2, and 3 illustrate some embodiments of a simple saccharide unit of ι-Carrageenan, κ-Carrageenan, and λ-Carrageenan, respectively. In some instances, only one type of carrageenan may be used for the treatment of a condition. For example, 76 -Carrageenan may be the only form of carrageenan administered to a subject. Nevertheless, in other embodiments, a plurality of carrageenan types may be used for the treatment of a condition. For example, ι-Carrageenan, κ-Carrageenan, and λ-Carrageenan could be administered in conjunction.

As indicated by the name polysaccharide, a carrageenan chain may have a length of many saccharide units. For example, a typical carrageenan chain can have a length of about a thousand saccharide units. As used herein, carrageenan may be of any suitable length, or a combination of varying lengths may be administered.

According to some embodiments, carrageenan may be may be co-administered with an effective amount of other agents that can further prevent and/or treat a condition. In some instances, these agents may act synergistically with carrageenan to increase the effectiveness of carrageenan. Some non-limiting examples of such agents may include polyamides, polyamide-like compounds, and/or surfactants. Indeed, carrageenan alone or carrageenan with one or more of the aforementioned agents can act as the active ingredient or ingredients (active ingredient(s)) in the described methods, systems, compositions, etc.

In some embodiments, carrageenan can be co-administered with any suitable polyamide and/or polyamide-like compound. Indeed, according to a non-binding theory, polyamides and polyamide-like compounds can interfere with viral DNA and viral replication while not interfering an organism's cellular genes. Moreover, the polyamides or polyamide-like compounds may help the described compositions to gain intracellular and extracellular access to target viral DNA so as to disrupt replication. Accordingly, when a polyamide or polyamide-like compound is co-administered with carrageenan, the two may better ameliorate, prevent, suppress, or otherwise treat a condition than either could separately. Although carrageenan may be co-administered with any suitable polyamide some non-limiting examples of suitable polyamides may include pyrole-containing polyamides (e.g., distamaycin, netropsin, and actinomycin). Similarly, while any suitable polyamide-like compound may be co-administered with carrageenan, some non-limiting examples of suitable polyamide-like compounds may include a pyrrole containing polyamide, N-methylpyrrole (Py), N-methylimidazole (Im), and N-mtheyl-3-hydroxypyrrole (Hp).

In some embodiments, carrageenan can be co-administered with any suitable surfactant agent. Generally, a surfactant agent can be used in disrupting non-covalent bonds in proteins, enhancing their negative charge to synergize the action of carrageenan, providing antisepsis in killing bacteria and other organisms, inhibiting oxidant reactions, and reducing mucosal irritation. Some non-limiting examples of suitable surfactants may include cetylpyridinium chloride (CPC), cocamidopropyl betaine (CAPB), and/or tyloxapol. For example, cocamidopropyl betaine (CAPB) may be used with carrageenan and/or another agent to reduce mucosal or vaginal irritation.

In some embodiments, any form of the active ingredient(s) can be administered to a subject. By way of non-limiting example, carrageenan and a polyamide may be administered in a crystalline, hydrated crystalline, amorphous form. Further, hydrates and solvates of the active ingredient(s) (e.g., carrageenan) may also be administered.

Similarly, in some embodiments, any derivative of the active ingredient(s), which includes constituents thereof, may be administered to a subject. For example, various stereochemical, tautomeric, recemic mixtures, geometric configuration, and/or mixtures thereof of active ingredient(s) may be administered. In another example, any desired functional group may be added to, subtracted form, and/or substituted for a functional group on the active ingredient(s).

In some embodiments, any prodrug for the active ingredient(s), or compound that can be transformed into the active ingredient(s) in vitro or in vivo, may be administered to a subject. Such a transformation may occur through various mechanisms, such as hydrolysis in the blood. For example, metabolites of carrageenan and/or a polyamide, a polyamide-like compound, and/or a surfactant may be formed by in vitro or in vivo through enzymatic and/or metabolic reactions.

Similarly, in some embodiments, a metabolite of the active ingredient(s) may be administered to a subject. This can be done by directly administering the metabolite to a subject or the metabolite can be administered by being produced in the subject through the subject's metabolism. For example, a metabolite of carrageenan and/or a polyamide, polyamide-like compound and/or a surfactant may be administered to a subject to treat a condition by administering the active ingredient(s) to the subject, after which administration, the desired metabolite is formed in the subject's body through metabolism. Additionally, the administration route and dosage of different forms and types of the active ingredient(s) can be varied, as desired, to obtain in vivo concentrations and rates of production of one or more metabolites of the active ingredient(s).

In some embodiments, the active ingredient(s), which, again may include one or more constituent components thereof, may be isotopically-labeled. In such instances, isotopically labeled agents and/or carrageenan chains may be identical to unlabeled agents and/or chains but for the fact that one or more atoms are replaced by an atom having an atomic mass or mass number different from the atomic mass or mass number usually found in nature. Examples of isotopes that can be incorporated into the active ingredient(s) may include isotopes of hydrogen, carbon, nitrogen, oxygen, phosphorous, sulfur, fluorine and chlorine, such as .sup.2H, .sup.3H, .sup.13C, .sup.14C .sup.15N, .sup.18O, .sup.17O, .sup.31P, .sup.32P, .sup.35S, .sup.18F, and .sup.36Cl, respectively. In some instances isotopically-labeled compounds, such as carrageenan into which radioactive isotopes (e.g., .sup.3H and .sup.14C) are incorporated, may be useful in drug and/or substrate tissue distribution assays. Tritiated, i.e., .sup.3H, and carbon-14, i.e., .sup.14C, isotopes may be preferred, particularly for their ease of preparation and detectability. Further, substitution with heavier isotopes such as deuterium (i.e., .sup.2H) may afford certain therapeutic advantages resulting from greater metabolic stability. For example, substitution with heavier isotopes may cause increased in vivo half-life or reduced dosage requirements and, hence, may be preferred in some circumstances. Isotopically labeled forms of the active ingredient(s) and/or constituents thereof may generally be prepared through procedures commonly known in the art.

As described herein, the active ingredient(s) may be administered to a subject in a variety of methods. For example, carrageenan alone or carrageenan along with a polyamide, polyamide-like composition, or surfactant may be administered to and/or taken by a subject rectally, vaginally, mucosally, transmucosally, submucosally, transurethrally, topically, intradermally, intraparetoneally, transdermally, etc. Thus, in some embodiments, the active ingredient(s) may be absorbed from the alimentary tract, whereas in other embodiments, the active ingredient(s) may be administered percutaneously or as a suppository.

In many cases, the route of administration may depend on the location (in or on the subject), nature and severity of the condition being treated as well as on the subject receiving treatment. No matter the route of administration, in some embodiments, the treatments with the active ingredient(s) may be conveniently presented in a unit dosage form and may be prepared by any conventional method. Several methods of administration are discussed below.

The active ingredient(s) may be administered in any form that allows the active ingredient(s) to prevent, suppress, ameliorate, or otherwise treat a condition. By way of non-limiting example, the active ingredient(s) can be in the form of a sterile, non-pyrogenic gel, liquid solution or suspension, foam, emulsion, aerosol, liquid capsule, coated capsule, suppository, aqueous cream, lyophilized powders, transdermal patch, polymeric or polymeric-like material and/or any other form known in the art, including any form suitable for known or novel pharmaceutical delivery systems or devices, such as a removable and/or absorbable, dissolvable, and/or degradable IVR.

In some embodiments, the active ingredient(s) may be administered in the form of a gel. In such embodiments, the gel may be applied in any suitable location (e.g., intravaginally or rectally. Because such a gel may provide sustained release of the active ingredient(s) over a prolonged period of time (e.g., 24-72 hours or longer), such a gel may be preferred for some applications. The gel may have any viscosity that is suitable for retaining it on the subject for a prolonged period of time. Similarly, the gel may be formulated to have a vaginally acceptable pH (e.g., from about 6.0 to about 8.0).

In some embodiments, capsules may be prepared by mixing the active ingredient(s) with a suitable diluent and filing the proper amount of the mixture in capsules. In such embodiments, any conventional diluent may be used for such capsules. For example, a suitable diluent, excipient, hyaluronate, or cellulosic compound may be included. Additionally, in some embodiments a capsule may also contain a liquid carrier such as fatty oil.

According to some embodiments, the active ingredient(s) may be used with a binder, including, but not limited to, polyethylene glycol, ethylcellulose, tragacanth, corn starch, and/or waxes. Additionally, in some embodiments, gels, creams, emulsions, suppositories, pills, capsules, and the like that contain the active ingredient(s) may also contain excipients, such as inert diluents, granulating agents, disintegrating agents, binding agents, and lubricating agents.

In some cases, the active ingredient(s) may be incorporated into an aqueous suspension in an admixture with excipients suitable for the manufacture of aqueous suspensions. Such excipients may be suspending, agents, for example, sodium carboxymethyl-cellulose, methylcellulose, hydroxy-propylmethycellulose, sodium alginate, polyvinyl-pyrrolidone, gum tragacanth, and gum acacia. Such excipients may also be dispersing or wetting agents like a naturally-occurring phosphatide (e.g., lecithin). The excipients may also be condensation products of an alkylene oxide with fatty acids, for example polyoxyethylene stearate. These excipients may also be condensation products of ethylene oxide with long chain aliphatic alcohols, for example heptadecaethylene-oxycetanol. Further, the excipients may be condensation products of ethylene oxide with partial esters derived from fatty acids and a hexitol, such as polyoxyethylene sorbitor monooleate. Additionally, these excipients may be condensation products of ethylene oxide with partial esters derived from fatty acids and hexitol anhydrides (e.g., polyethylene sorbitan monooleate). These excipients may also be the sodium salt of hyaluronic acid of various molecular weights.

In some embodiments, the active ingredient(s) may be administered and/or taken as a suppository. In this manner, the active ingredient may be administered locally to an infected part of the body (e.g., rectally, vaginally, or transurethrally). In some cases, any conventional suppository base may be used when the active ingredient(s) are administered as a suppository. For example, cocoa butter is a traditional suppository base, which may be modified by addition of waxes to raise its melting point slightly. In another example, water-miscible suppository bases may be used, including, but not limited to, polyethylene glycols of various molecular weights and/or sodium hyaluronate of various molecular weights.

In some embodiments, the active ingredient(s) may be administered topically and/or transdermally. There are many conventional methods of topical and transdermal administration, and any type or combination of those methods may be used for the administration of the active ingredient(s). For example, the active ingredient(s) may be applied as a topical ointment. In another example, the active ingredient(s) may be applied in a gel, lotion or cream. In yet another example, the active ingredient(s) could be mixed with a substance like talcum and be applied to the subject's skin. In still another example, the active ingredient(s) may be administered to a subject by the use of a transdermal patch. Although any conventional transdermal patch and method of use may be implemented, some common transdermal patches may include single-layer drug-in-adhesive, multi-layer drug-in-adhesive, reservoir, and matrix transdermal patches. Additionally, in some embodiments, the active ingredient(s) may be combined with one or more substances, like alcohol or sodium hyaluronate of various molecular weights, which may increase the ability of the active ingredient to penetrate a subject's skin or tissue.

In some implementations, the active ingredient(s) may be administered intravaginally or intrauterinally through any known or novel apparatus, device, system, and/or composition that is suitable for delivering the active ingredient(s). For example, an IVR, similar to the NOVARING®, or an IVR comprising a polymeric or polymeric-like material, may contain or be coated with a form of the active ingredient(s). In this example, the active ingredient(s) may be released from the ring over an extended period of time. For example, an IVR may release active ingredient(s) in a sustained, controlled, continuous, and/or prolonged manner. Indeed, in some embodiments, the IVR may release active ingredient(s) for a period of time as short as 1 minute. In other embodiments, however, the IVR may release active ingredient(s) for a period of time as long as a year. Nevertheless, in some preferred embodiments, the IVR may release active ingredients for approximately 1 day to 2 months. In other preferred embodiments, the IVR can release the active ingredients for approximately one month or about 30 days. Such a device may use any method known of coating or method for slowing the release of an active ingredient(s) (e.g., the active ingredient(s) may be integrated into a matrix or a fat soluble material). In this manner, a subject may be able to prevent or treat a condition, such as HPV over a sustained period of time with little effort.

In another example, an apparatus, such as a tampon, feminine pad, or tissue may be coated and/or impregnated with the active ingredient(s) and, thereby, be used to prevent/treat/suppress and/or ameliorate a condition in a simple and inexpensive manner. In still another example, the active ingredient(s) may be integrated into a cleanser, such as soap, shampoo, douche, or a yeast infection kit. In this manner, the active ingredient(s) may be used as desired with little extra effort.

In some embodiments, the effects of the active ingredient(s) may be further delayed or prolonged. Such delays may be accomplished using any method known in the art. For example, a slowly soluble gel the active ingredient(s) may be prepared and or a slowly soluble pellet of the active ingredient(s) may be incorporated in a tablet or capsule. This technique for delaying release may be improved by making and incorporating bases, pellets, gels, etc. of several different dissolution rates. For example, a suppository comprising several bases with different dissolution rates may be used to prolonged release of the active ingredient(s).

In each of the above methods of administration, the different gels, creams, lotions, sprays, suppositories, foams, aerosols, solutions, capsules, transdermal patches, IVRs and so forth may be formulated to contain any desired concentration of the active ingredient(s). No matter the method of administration, the dosage of active ingredient(s) administered to a subject may be any desired amount or concentration; preferably an effective amount will be used. As used herein, an effective amount of the active ingredient(s) may simply be any amount of the active ingredient(s) that is capable of treating/preventing/suppressing and/or ameliorating a condition. The specific dose of the active ingredient(s) administered according to a subject may, of course, be determined by the particular circumstances surrounding the case including, for example, the active ingredient(s) administered (e.g., carrageenan alone or carrageenan with a polyamide), the form of the active ingredient(s) administered (e.g., gel, suppository, etc.), the route of administration, the state of being of the subject, and the severity of the pathological condition being treated. Thus, the administration route and dosage of the active ingredient(s) can be modified, as desired, to obtain desired in vivo concentrations.

In some embodiments, the active ingredient(s) may be prepared and formulated and to be delivered in a dose for a specified period (e.g., a daily dose, a fraction of a daily dose, a weekly dose, a monthly dose, or a one time dose). Nevertheless, in other embodiments, no particular dosage need be formulated and the active ingredient(s) containing composition of device may be used as desired. For example, a gel containing a daily dose of active ingredient(s) can be administered daily. In another example, an IVR comprising a monthly dose of the active ingredient(s) can be removed and replaced monthly.

Further, the active ingredient(s) and compounds or devices containing the active ingredient(s) may also be administered in any effective time frame. An effective time frame may be any time that allows the active ingredient(s) to treat/prevent/suppress and/or ameliorate a condition. For example, the active ingredient(s) may be administered to a subject that appears to be free from a condition. In this example, the active ingredient(s) may, thus, be used prophylacticly to prevent the subject from catching a condition, such as HPV or cervical cancer. In another example, the active ingredient(s) could be used before coitus to prevent the spread of a sexually transmitted disease, such as HPV. In another example, the active ingredient(s) and compositions containing the active ingredient(s) may be taken once a subject has a condition. In this example, the active ingredient(s) may be used to suppress, treat, and/or ameliorate the condition as well as prevent it from spreading to another.

Also, the active ingredient(s) may also be administered any number of times. For example, the active ingredient(s) could be administered once or the active ingredient(s) may be administered, potentially, thousands of times. In any case, the number of times the active ingredient(s) are administered may depend on the subject (e.g., age, size, sex, temperament, culture, financial status, etc), the type of the condition, the severity of the condition, and so forth.

In some embodiments, the various active ingredient(s) may be co-administered with other drugs, pharmaceuticals, nutraceuticals, supplement, chemicals, substances, and/or compounds. Any drug, nutraceutical, chemical, substance, and/or compound that may be used for the treatment/prevention/suppression or amelioration of the condition(s) for which the active ingredient(s) may be useful may also be co-administered with the active ingredient(s). In one example, an antiviral agent may be co-administered with the active ingredient(s). In another example, other compounds that may mimic heparin sulfate and, thereby, compete against virons for initial attachment to cell surfaces may be used. Some examples of such mimics may include, but are not limited to, cellulose sulfate, dextran sulfate, heparin sulfate, heparin, chondroitin 4-sulfate, chondroitin 6-sulfate, and/or any sulfated polysaccharide.

In another example, the active ingredient(s) could be co-administered with any suitable defensin, such as human α-defensins 1, 2, 3, and 5. In yet another example, the active ingredient(s) may be co-administered with any pain killer, anesthetic, steroid, antimicrobial, anti-pyretic, antibiotic, anti-fungal, antioxidant, and/or an anti-cholesterimic.

As mentioned, the active ingredient(s) may be co-administered with any food supplement or nutraceutical. Some examples, of administrable food supplements and/or nutraceuticals may include any mineral, vitamin, herb, botanical, amino acid, ginseng, ginger, St. John's wart, noni, Echinacea, and so forth.

In some embodiments, the active ingredient(s) may be co-administered with any other desired substance in any conventional method of co-administration. For example, the active ingredient(s) can be co-administered together with another substance as a composition or as part of the same, unitary dosage form. Additionally, in some embodiments, one or more of the active ingredient(s) can be administered with another substance separately, but as part of the same therapeutic treatment program or regimen. In such a case, the components need not necessarily be administered at essentially the same time, although they can be if so desired. Thus co-administration may also include, for example, administering carrageenan, one or more agents, and/or an additional compound as separate dosages or dosage forms, but at the same time. Furthermore, co-administration may also include separate administration at different times and in any order. For example, where appropriate a subject may take one or more component(s) of the treatment in the morning and the one or more of the other component(s) at night.

Additionally, in some embodiments, the described methods and systems may also include kits for use by a subject for treating a condition. Typically, a kit may comprise a) a pharmaceutical composition comprising the active ingredient(s) and a pharmaceutically acceptable carrier, vehicle or diluent; and, optionally, b) instructions describing a method of using the pharmaceutical composition for treating a specific condition. In some embodiments, the instructions may also indicate that the kit is for treating certain conditions, while substantially reducing the concomitant liability of adverse effects that may be associated with the administration of active ingredients.

In some embodiments, a kit may also include a container for containing the separate unit dosage forms, such as a divided bottle, a syringe like device, or a disposable pre-filled applicator, or a divided foil packet or equivalent. For example, a kit may comprise an applicator (e.g., a prefilled disposable applicator or a prefilled applicator with a plunger-type mechanism) for convenient application of a gel containing the active ingredient(s).

Where the kit comprises a container, the container can be in any conventional shape or form as known in the art which is made of a pharmaceutically acceptable material, for example a sealed packet (e.g., foil), a paper or cardboard box, a glass or plastic bottle or jar, a re-sealable bag (e.g., to hold a “refill” of tablets for placement into a different container), or a blister pack with individual doses for pressing out of the pack according to a therapeutic schedule. The container employed may depend on the exact dosage form involved, for example a conventional cardboard box may not generally be used to hold a gel or liquid suspension. Further, it is feasible that more than one container may be used together in a single package to market a single dosage form. For example, foil packets containing gel may be contained within a box, which in turn may be wrapped in a plastic film.

An example of such a kit is a so-called foil packet. Foil packets are well known in the packaging industry and are being widely used for the packaging of pharmaceutical unit dosage forms (gels, tablets, capsules, and the like). Foil packets generally consist of two sheets of foil that are joined together to form a sealed cavity. The cavity may have the size and shape of individual dose form of gel or cream. Next, the dosage form of active ingredient(s) may be placed in the cavity and the packet may be sealed. As a result, dosage forms may be individually sealed or collectively sealed, as desired, in the cavity. Preferably the strength of the foil sheets is such that the dose form may be removed from the packet by manually tearing the packet and applying pressure on the cavity. In this example, the gel may then be squeezed out of the opening.

In some embodiments, it may be desirable to provide a written memory aid, where the written memory aid is of the type containing information and/or instructions for the physician, pharmacist or subject, e.g., in the form of number of times a day or week a gel should be applied. Another example of such a memory aid may be a calendar printed on the card e.g., as follows “First Week, Monday, Tuesday, . . . ” etc. “Second Week, Monday, Tuesday, . . . ” etc. Additionally, any other variations of memory aids may be implemented.

In another embodiment, the kit may be a dispenser designed to dispense the daily doses one at a time. In some embodiments, the dispenser may be equipped with a memory-aid, so as to further facilitate compliance with the regimen. An example of such a memory-aid may be a mechanical counter, which indicates the number of daily doses that, have been dispensed. Another example of such a memory-aid may be a battery-powered micro-chip memory coupled with a liquid crystal readout, or audible reminder signal which, for example, reads out the date that the last daily dose has been taken and/or reminds one when the next dose is to be taken.

In addition to any previously indicated modification, numerous other variations and alternative arrangements may be devised by those skilled in the art without departing from the spirit and scope of the invention, and appended claims are intended to cover such modifications and arrangements. Thus, while the invention has been described above with particularity and detail in connection with what is presently deemed to be the most practical and preferred aspects of the invention, it will be apparent to those of ordinary skill in the art that numerous modifications, including, but not limited to, form, function, manner of operation and use may be made without departing from the principles and concepts set forth herein. Also, as used herein, examples are meant to be illustrative only and should not be construed to be limiting in any manner.

1. A method of treating an ailment with carrageenan, the method comprising:
administering an effective amount of carrageenan to a subject.

2. The method of claim 1, wherein the carrageenan comprises ι-Carrageenan, κ-Carrageenan, λ-Carrageenan, or mixtures thereof.

3. The method of claim 1, wherein the carrageenan comprises a carrageenan metabolite.

4. The method of claim 1, wherein the carrageenan comprises a carrageenan prodrug.

5. The method of claim 1, where the carrageenan is co-administered with an effective amount of an agent selected from the following:
(a) a polyamide,
(b) a pyrrole containing polyamide, and
(c) a surfactant.

6. The method of claim 5, wherein the polyamide is selected from distamaycin, netropsin, and actinomycin.

7. The method of claim 5, wherein the pyrrole containing polyamide is selected from N-methylpyrole (Py), N-methylimidazole (Im), and N-mtheyl-3-hydroxypyrrole (Hp).

8. The method of claim 5, wherein the method further comprises co-administering the carrageenan and the agent as a suppository, intravaginally, transdermally, topically, rectally, transurethrally, or a combination thereof.

9. The method of claim 5, wherein carrageenan and the agent are co-administered via a transdermal patch.

10. The method of claim 5, wherein the carrageenan and the agent are co-administered via an intrauterine device or an intravaginal ring

11. The method of claim 5, wherein the carrageenan and the agent are co-administered by an intrauterine device or an intravaginal ring that is absorbed by the subject.

12. The method of claim 5, wherein the ailment is selected from human papillomavirus, human immunodeficiency virus, or herpes simplex virus.

13. The method of claim 12, wherein the ailment comprises human papillomavirus.

14. The method of claim 5, wherein the carrageenan and the agent are co-administered in an aqueous or solid/soluble gel to provide a controlled release of the carrageenan and the agent, wherein the gel is adapted to be administered using an applicator.

15. The method in claim 10, wherein use of intravaginal device comprises an intravaginal polymeric type ring that provides a controlled release of the carrageenan and the agent.

16. The method in claim 15, wherein the intravaginal polymeric type ring releases the carrageenan and the agent to the subject from about one day to about one month.

17. The method in claim 5, wherein the carrageenan and the agent both prevents and treats human papillomavirus.

18. The method of claim 5, wherein the carrageenan and the agent are co-administered with at least one of the following: a pain killer, an anesthetic, a steroid, an antimicrobial, an anti-pyretic, an antibiotic, an anti-fungal, an antioxidant, an anti-cholesterimic.

19. A kit for treating human papillomavirus, the kit containing a pharmaceutically effective amount of carrageenan and an agent selected from a polyamide, a polyamide-like compound, and a surfactant.

20. A method for treating human papillomavirus, comprising:
providing a pharmaceutically effective amount of carrageenan;
providing a pharmaceutically effective amount of an agent selected from a polyamide, a polyamide-like compound, and a surfactant; and
administering a portion thereof to a subject.
